(19) United States
(12) Patent Application Publication
(10) Pub. No.: US 2026/0151225 A1
(43) Pub. Date: Jun. 4, 2026

(54) CARDIAC VALVE WITH EXPANDABLE MATERIAL DISPOSED ON SKIRT

(71) Applicant: BOSTON SCIENTIFIC SCIMED, INC., MAPLE GROVE, MN (US)

(72) Inventors: Joshua Stephan Havel, Champlin, MN (US); Kent Grove, Sartell, MN (US); Chenhao Fu, Plymouth, MN (US)

(73) Assignee: BOSTON SCIENTIFIC SCIMED, INC., MAPLE GROVE, MN (US)

(21) Appl. No.: 19/406,231

(22) Filed: Dec. 2, 2025

Related U.S. Application Data
(60) Provisional application No. 63/726,737, filed on Dec. 2, 2024.

(51) Int. Cl. A61F 2/24

(52) U.S. Cl. CPC A61F 2/2418; A61F 2210/0071

(57) ABSTRACT

An implantable medical device that is adapted to be implanted at an implantation site includes an expandable frame that is adapted to expand from a collapsed configuration to an expanded configuration. The expandable frame includes an upper crown portion, a lower crown portion, commissural posts that extend proximally from the upper crown portion, and stabilization arches that extend proximally from the commissural posts. One or more valve cusps are secured relative to the expandable frame. An inner skirt is disposed along an inner portion of the lower crown portion and an outer skirt is disposed along an outer portion of the lower crown portion of the expandable frame. An expandable member is secured to the outer skirt.

### CROSS REFERENCE TO RELATED APPLICATIONS

This application claims the benefit of priority of U.S. Provisional Application No. 63/726,737 filed Dec. 2, 2024, the entire disclosure of which is hereby incorporated by reference.

### TECHNICAL FIELD

The disclosure relates generally to medical devices and more particularly to replacement cardiac valves.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in an implantable medical device that is adapted to be implanted at a treatment site. The implantable medical device includes an expandable frame that is adapted to expand from a collapsed configuration to an expanded configuration. The expandable frame includes an upper crown portion and a lower crown portion. An inner skirt is disposed along an inner portion of the lower crown portion of the expandable frame and an outer skirt is disposed along an outer portion of the lower crown portion of the expandable frame. An expandable member is secured to the outer skirt.

Alternatively or additionally, the expandable member may include a plurality of foam elements that are secured to the outer skirt such that the plurality of foam elements are disposed between the outer skirt and inner skirt.

Alternatively or additionally, the outer skirt may include an inner surface facing towards the lower crown portion of the expandable frame and an opposing outer surface, and the plurality of foam elements may be printed onto the inner surface.

Alternatively or additionally, the inner skirt and the outer skirt may both be part of a polymeric fold-over skirt.

Alternatively or additionally, the polymeric fold-over skirt may be positioned folded over a lower extent of the lower crown portion of the expandable frame, with a portion of the polymeric fold-over skirt inside of the lower crown portion of the expandable frame forming the inner skirt and a portion of the polymeric fold-over skirt outside of the lower crown portion of the expandable frame forming the outer skirt.

Alternatively or additionally, the polymeric fold-over skirt may include PET (polyethylene terephthalate).

Alternatively or additionally, the lower crown portion may include a plurality of struts.

Alternatively or additionally, each of the plurality of foam elements may extend radially beyond individual struts of the plurality of struts.

Alternatively or additionally, each of the plurality of foam elements may span circumferentially across individual struts of the plurality of struts.

Another example may be found in a replacement cardiac valve that is adapted to be implanted within a native cardiac valve annulus. The replacement cardiac valve includes an expandable frame that is adapted to expand from a collapsed configuration to an expanded configuration. The expandable frame includes an upper crown portion and a lower crown portion that includes a plurality of struts. A composite skirt is wrapped over a lower extent of the lower crown portion. The composite skirt includes an inner skirt that is disposed inside the lower crown portion and an outer skirt that is disposed outside of the lower crown portion. A plurality of foam elements are secured to the outer skirt.

Alternatively or additionally, the composite skirt may include PET (polyethylene terephthalate).

Alternatively or additionally, the plurality of foam elements may be disposed between the inner skirt and the outer skirt.

Alternatively or additionally, the plurality of foam elements may be adapted to remain outside of the plurality of struts of the lower crown portion.

Alternatively or additionally, the plurality of foam elements may be adapted to extend into spaces between struts of the plurality of struts of the lower crown portion.

Another example may be found in a replacement cardiac valve that is adapted to be implanted within a native cardiac valve annulus. The replacement cardiac valve includes an expandable frame that is adapted to expand from a collapsed configuration to an expanded configuration. The replacement cardiac valve includes an inner skirt that surrounds a portion of the expandable frame, an outer skirt that surrounds a portion of the expandable frame, and a plurality of foam elements that are secured to the outer skirt.

Alternatively or additionally, the expandable frame may include an upper crown portion, a lower crown portion including a plurality of struts, commissural posts that extend proximally from the upper crown portion, and stabilization arches that extend proximally from the commissural posts.

Alternatively or additionally, the outer skirt may include an inner surface that faces the expandable frame and an outer surface that faces away from the expandable frame. The plurality of foam elements may be secured to the inner surface of the outer skirt.

Alternatively or additionally, the outer skirt may include a polymeric outer skirt.

Alternatively or additionally, the outer skirt may include PET (polyethylene terephthalate).

Alternatively or additionally, the plurality of foam elements may extend towards the inner skirt but may not be attached to the inner skirt.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an illustrative expandable frame;

FIG. 2 is a side view of an illustrative replacement cardiac valve including the illustrative expandable frame of FIG. 1;

FIG. 3 is a side view of an illustrative replacement cardiac valve;

FIG. 4 is a top view of the illustrative replacement cardiac valve of FIG. 3;

FIG. 5 is a cross-sectional view taken along the line 5-5 of FIG. 3;

FIG. 6 is a side view of an illustrative replacement cardiac valve;

FIG. 7 is a top view of the illustrative replacement cardiac valve of FIG. 6; and

FIG. 8 is a cross-sectional view taken along the line 8-8 of FIG. 6.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

In some instances, an implantable medical device is adapted to be implanted at a treatment site. The implantable medical device includes an expandable frame that is adapted to expand from a collapsed configuration to an expanded configuration. The expandable frame includes an upper crown portion, a lower crown portion, commissural posts that extend proximally from the upper crown portion, and stabilization arches that extend proximally from the commissural posts. One or more valve cusps are secured relative to the expandable frame. An inner skirt is disposed along an inner portion of the lower crown portion of the expandable frame and an outer skirt is disposed along an outer portion of the lower crown portion of the expandable frame. An expandable member is secured to the outer skirt.

In some cases, the expandable member may include a plurality of foam elements that are secured to the outer skirt such that the plurality of foam elements are disposed between the outer skirt and inner skirt. The outer skirt may include an inner surface facing towards the lower crown portion of the expandable frame and an opposing outer surface, and the plurality of foam elements may be printed onto the inner surface. In some cases, the foam elements may be 3D printed onto the inner surface of the outer skirt, while the outer skirt is held in a flat, 2D configuration. After printing, the outer skirt may be rolled into a tubular configuration. In some cases, a tubular shape may be formed, and then the foam may be deposited directly on the tubular shape as the tubular shape is rotated on a collet. The tubular configuration may then be inverted to form the outer skirt with the foam elements disposed on the inner surface of the outer skirt. In some cases, the inner skirt and the outer skirt may both be part of a polymeric fold-over skirt. In some cases, the polymeric fold-over skirt may be positioned over a lower extent of the lower crown portion of the expandable frame such that a portion of the polymeric fold-over skirt inside of the lower crown portion of the expandable frame forms the inner skirt and a portion of the polymeric fold-over skirt outside of the lower crown portion of the expandable frame forms the outer skirt. In some cases, the polymeric fold-over skirt may include PET (polyethylene terephthalate). In some cases, the lower crown portion may include a plurality of struts. In some cases, each of the plurality of foam elements may extend radially beyond individual struts of the plurality of struts. In some cases, each of the plurality of foam may span circumferentially across individual struts of the plurality of struts.

In some instances, a replacement cardiac valve is adapted to be implanted within a native cardiac valve annulus. The replacement cardiac valve includes an expandable frame that is adapted to expand from a collapsed configuration to an expanded configuration. The expandable frame includes an upper crown portion, a lower crown portion including a plurality of struts, commissural posts that extend proximally from the upper crown portion, and stabilization arches that extends proximally from the commissural posts. A composite skirt is wrapped over a lower extent of the lower crown portion. The composite skirt includes an inner skirt that is disposed inside the lower crown portion and an outer skirt that is disposed outside of the lower crown portion. A plurality of foam elements are secured to the outer skirt.

In some cases, the composite skirt may include PET (polyethylene terephthalate). The plurality of foam elements may be disposed between the inner skirt and the outer skirt. In some cases, the plurality of foam elements may be adapted to remain outside of the plurality of struts of the lower crown portion. In some cases, the plurality of foam elements may be adapted to extend into spaces between struts of the plurality of struts of the lower crown portion.

In some instances, a replacement cardiac valve is adapted to be implanted within a native cardiac valve annulus. The replacement cardiac valve includes an expandable frame that is adapted to expand from a collapsed configuration to an expanded configuration, the expandable frame including a lower extent. One or more valve cusps are secured relative to the expandable frame. An inner skirt surrounds a portion of the expandable frame and an outer skirt surrounds a portion of the expandable frame. A plurality of foam elements are secured to the outer skirt.

In some cases, the expandable frame may include an upper crown portion, a lower crown portion including a plurality of struts, commissural posts that extend proximally from the upper crown portion, and stabilization arches that extend proximally from the commissural posts. In some cases, the outer skirt may include an inner surface facing the expandable frame and an outer surface facing away from the expandable frame. The plurality of foam elements may be secured to the inner surface of the outer skirt. The outer skirt may include a polymeric outer skirt. The outer skirt may include PET (polyethylene terephthalate). In some cases, the plurality of foam elements may extend towards the inner skirt but may not be attached to the inner skirt.

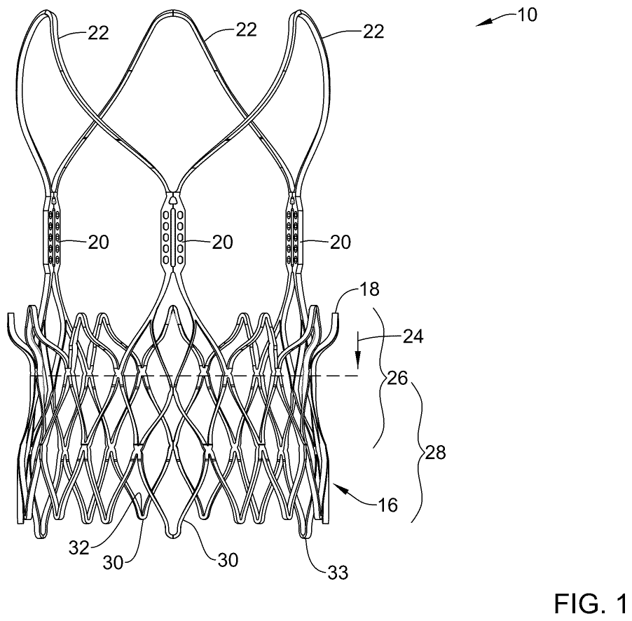

FIG. 1 is a side view of an illustrative replacement cardiac valve expandable frame 10. The expandable frame 10 may be used as a starting point for assembly of a replacement aortic valve, a replacement mitral valve, a replacement pulmonary valve or a replacement tricuspid valve, for example. Some non-limiting examples of the replacement cardiac valve may include the ACURATE NEO2™, the ACURATE PRIME™, and/or family members thereof from Boston Scientific. Other examples are also contemplated. In use, the replacement cardiac valve may be implanted (e.g., surgically or through transcatheter delivery) in a mammalian heart. The replacement cardiac valve can be configured to allow one-way flow through the replacement cardiac valve from an inflow end to an outflow end. In some cases, the resulting replacement cardiac valve may include biological tissue such as porcine or bovine pericardium and/or natural cardiac valve leaflets such as natural porcine cardiac valve leaflets that are secured relative to the replacement cardiac valve expandable frame 10. In some cases, the natural cardiac valve leaflets may be attached to a portion of natural cardiac wall tissue. The biological material may be fixed, for example, using glutaraldehyde.

The expandable frame 10 may be compressible to a radially compressed, or collapsed, configuration for delivery using a delivery catheter, and may be expandable to an expanded configuration (as shown) during implantation. In some cases, the expandable frame 10 may be self-expanding. In some embodiments, the expandable frame 10 may be self-biased toward the radially expanded configuration. In some embodiments, the expandable frame 10 may be mechanically expandable. In some embodiments, the expandable frame 10 may be balloon expandable. Other configurations, including combinations thereof, are also contemplated. In some embodiments, the expandable frame 10 may have a substantially circular cross-section. In some embodiments, the expandable frame 10 can have a non-circular (e.g., D-shaped, elliptical, etc.) cross-section. Some suitable but non-limiting examples of materials that may be used to form the expandable frame 10, including but not limited to metals and metal alloys, composites, ceramics, polymers, and the like, are described below. In some cases, the expandable frame 10 may include a lower tubular or crown portion 16 proximate and/or at an inflow end, an upper crown portion 18 proximate and/or at an outflow end, a plurality of upstanding commissural posts 20, and a plurality of stabilization arches 22 extending downstream from the outflow end. In use, the lower portion 16 of the expandable frame 10 may be adapted to be deployed after the other regions of the expandable frame 10. For example, the arches 22, the commissural posts 20 and the upper crown 18 may be deployed at least partly before the lower portion 16 (in that order, or in reverse order, or in a different order). At the very least, once the upper crown 18 has been at least partly deployed, the expandable frame 10 may be urged and/or displaced in the direction of arrow 24 to seat the upper crown 18 against native leaflets at the treatment site. In this example, deploying the lower portion 16 last fixes the expandable frame 10 in its final position.

The lower portion 16, and optionally a portion of the upper crown 18, may be formed by a lattice structure of the stent that is formed by a plurality of struts 30 that form generally diamond-shaped apertures or cells 32. It will be appreciated that the cells 32 are generally diamond-shaped when the expandable frame 10 is in its expanded configuration, as shown in FIG. 1. The cells 32 will generally disappear as the struts 30 collapse down onto each other when the expandable frame 10 is in its collapsed configuration. In some cases, the lower portion 16 may be considered as including a lower extent 33. In some cases, the native leaflets may generally overlap a portion 26 of the expandable frame 10. The native valve annulus may overlap a portion 28 of the expandable frame 10.

The expandable frame 10 may optionally be of a self-expanding type that is compressible to the compressed configuration for loading into a delivery catheter for delivery to the site of implantation. In use, by removal of the constraining effect of a sheath holding the expandable frame 10 in the compressed configuration, the expandable frame 10 self-expands to or towards the operative configuration. A self-expanding stent may, for example, be of shape-memory material, for example, shape-memory metal alloy, for example, nitinol. Alternatively, the expandable frame 10 may be configured to be expanded by application of a foreshortening force from the delivery catheter and/or by application of expanding force from the delivery catheter, such as by using an expansion balloon. These are just examples.

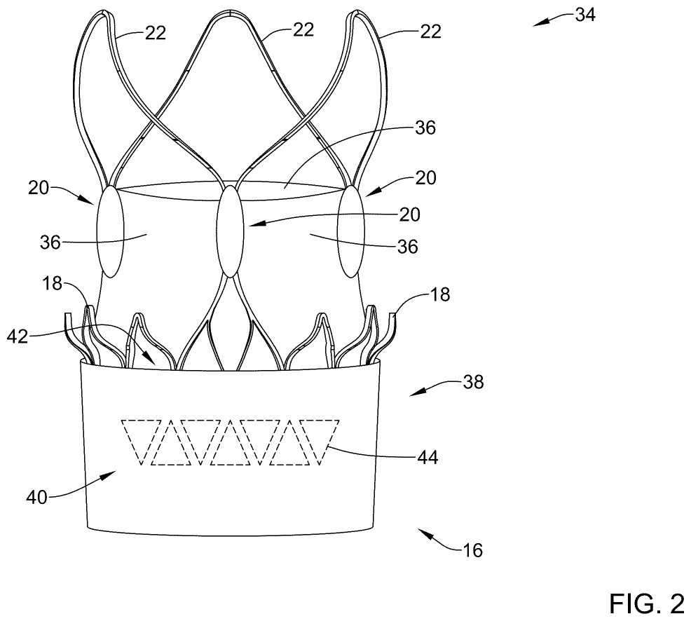

FIG. 2 is a side view of an illustrative replacement cardiac valve 34 that may be constructed using the expandable frame 10 that was discussed with respect to FIG. 1. The replacement cardiac valve 34, includes several valve cusps 36. As shown, the replacement cardiac valve 34 includes a total of three valve cusps 36. Each of the valve cusps 36 are secured to the expandable frame 10. In some cases, each of the valve cusps 36 are secured in part by being sutured to the commissure posts 20 and in part by being sutured to portions of the expandable frame 10 within the portion 26 of the expandable frame 10. The replacement cardiac valve 34 also includes a composite skirt 38 that extends over the portion 28 of the expandable frame 10. In some cases, the composite skirt 38 may include an outer skirt 40 and an inner skirt 42. In some cases, the outer skirt 40 may extend about an exterior of the expandable frame 10 and the inner skirt 42 may extend about an interior of the expandable frame 10.

In some cases, one or more expandable members 44 may be placed between the outer skirt 40 and the inner skirt 42. In some cases, the one or more expandable members 44 may represent a hydrogel. In some cases, the one or more expandable members 44 may represent a specifically shaped material that fills a volume. In some cases, the one or more expandable members 44 may be foam elements that are secured to the outer skirt 40. In some cases, the foam elements may be printed onto the outer skirt 40. To form the foam elements, a 3D printer would receive a recipe (STL file for example) that has the shape of the desired elements which would then be printed on the outer skirt 40. The foam would be deposited in thin layers that build on top of each other to create the height of the elements. The total volume would be constrained between what can be reasonably loaded onto a delivery system and pushed through an introducer and what provides the best sealing potential. All shapes could be used in order to push the outer skirt 40 out and fill the pockets (rods, cones, spheres, blocks, stent contouring geometries, etc.). The expandable members 44 may be placed in a continuous or discontinuous pattern that spans circumferentially across the individual struts of the plurality of struts 30 on the expandable frame 10 along the outer skirt 40 depending on the density of the foam. Use of specific patterning may impact the delivery of the cardiac valve. Sizes of these elements may vary. In some cases, foam elements may be about 4 or 5 millimeters high from the skirt and may fill the area between open stent cells.

The foam elements can be highly compressed in order to facilitate compressing the replacement cardiac valve 34 into a compressed configuration for delivery. The foam elements can expand considerably when freed from the compressive forces holding the replacement cardiac valve 34 into the compressed configuration for delivery. A variety of different polymeric materials may be used for forming the foam elements.

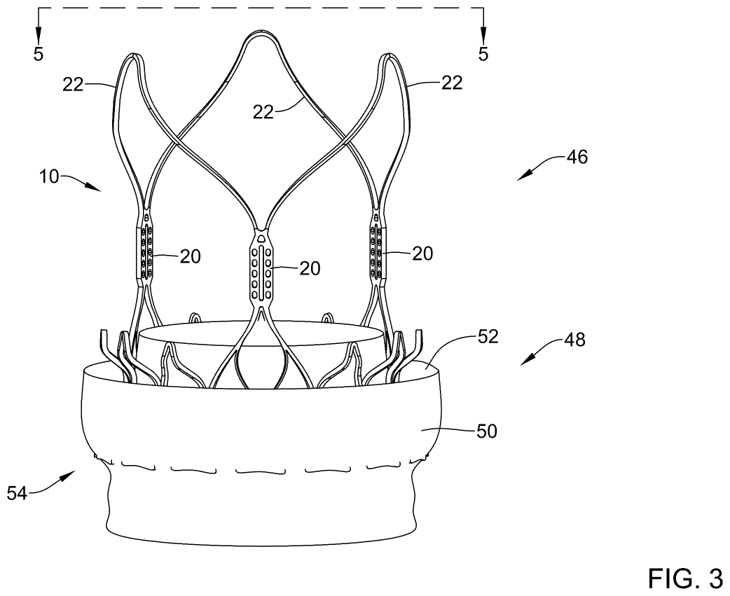

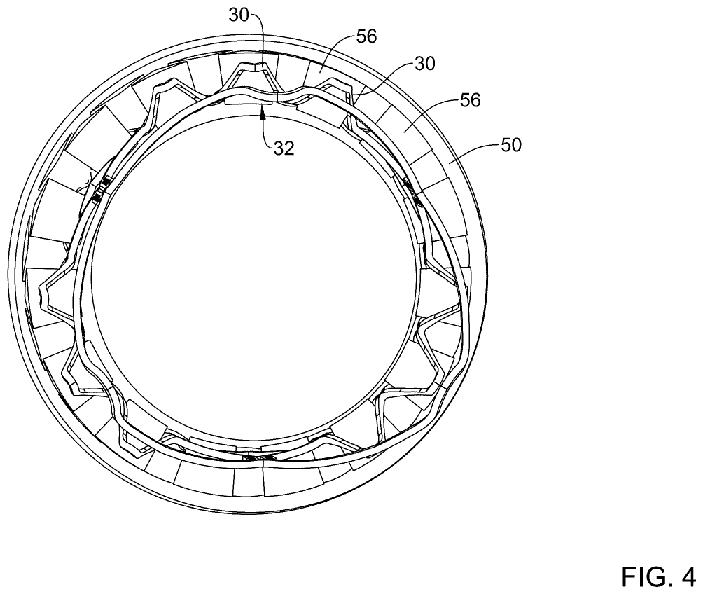

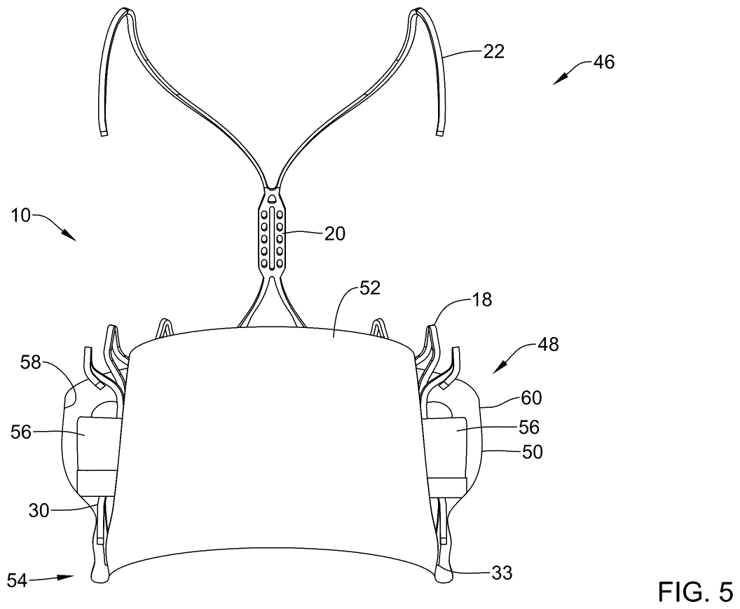

FIG. 3 is a side view of an illustrative replacement cardiac valve 46 that is assembled using the expandable frame 10 that was discussed with respect to FIG. 1. FIG. 4 is a top view of the replacement cardiac valve 46 and FIG. 5 is a cross-sectional view of the replacement cardiac valve 46 taken along the line 5-5 of FIG. 3. The replacement cardiac valve 46 includes a composite skirt 48 that includes an outer skirt 50 and an inner skirt 52. In some cases, the composite skirt 48 may be a polymeric fold-over skirt 54 that wraps around the lower extent 33 of the expandable frame 10 such that a portion of the polymeric fold-over skirt 54 extending outside of the expandable frame 10 forms the outer skirt 50 and a portion of the polymeric fold-over skirt 54 extending inside of the expandable frame 10 forms the inner skirt 52. FIG. 5 shows how the polymeric fold-over skirt 54 wraps or bends around the lower extent 33 of the expandable frame 10. In some cases, having the outer skirt 50 and the inner skirt 52 formed from a single sheet of polymer improves entrapment of fluids to reduce or eliminate the occurrence of paravalvular leak or replacement valve regurgitation. A variety of different polymers may be used to form the polymeric fold-over skirt 54. In some cases, the polymeric fold-over skirt 54 may be formed of PET (polyethylene terephthalate). Additional examples include UHMWPE (Ultra High Molecular Weight Polyethylene), TPU (thermoplastic polyurethane), PI (polyimide), PCU (polycarbonate urethane), SiPUU (siloxane-based polyurethane-urea), SIBS (poly (styrene-block-isobutylene-block-styrene), xSIBS (cross-linked SIBS), SEBS (styrene-ethylene-butylene-styrene), Triblock polyurethane combining siloxane and carbonate, ePTFE (expanded polytetrafluoroethylene), PTFE (polytetrafluoroethylene), or biodegradable materials. In some cases, the polymeric fold-over skirt 54 may be made of a polymer that is biocompatible with low thrombogenicity.

The replacement cardiac valve 46 includes a plurality of foam elements 56 that are disposed between the outer skirt 50 and the inner skirt 52. In some cases, the outer skirt 50 may be considered as including an inner surface 58 that faces towards the expandable frame 10 and an outer surface 60 that faces away from the expandable frame 10. In some cases, the foam elements 56 may each be secured to the inner surface 58 of the outer skirt 50. In some cases, the foam elements 56 may be adhesively secured to the inner surface 58 of the outer skirt 50. The foam elements 56 may each be printed onto the inner surface 58 of the outer skirt 50 and may extend towards the inner skirt 52 and may even contact the inner skirt 52, but may not be secured to the inner skirt 52. In some cases, the foam elements 56 extend radially beyond individual struts 30 and thus extend through the diamond-shaped apertures or cells 32. In some cases, the foam elements 56 may be shaped to be complementary in shape to the diamond-shaped apertures or cells 32. In some cases, the foam elements 56 may be triangular-shaped, such that some of the foam elements 56 extend into an upper half of a corresponding diamond-shaped aperture or cell 32, and some of the foam elements 56 extend into a lower half of a corresponding diamond-shaped aperture or cell 32. When the replacement cardiac valve 46 is in its expanded configuration (as shown), the foam elements 56 help to push the outer skirt 50 into contact with the native annulus, and can help to seal around calcium deposits within the native annulus. In some cases, the foam elements 56 help to push the outer skirt 50 away from the inner skirt 52, which helps to form a pocket that better catches the diastolic pressure and causes the outer skirt 50 to further expand away from the inner skirt 52. Dimensions for the foam elements 56 may be selected to work with the particular dimensions of the expandable frame 10, for example. In some cases, the addition of the foam elements 56 may assist in better distributing forces from the expandable frame 10 that act on the anatomy, including the native annulus, and thus may reduce permanent pacemaker rates. In some cases, addition of the foam elements 56 may reduce migration of the replacement cardiac valve 46 by increasing the surface area by which pressure is applied to the anatomy by the replacement cardiac valve 46.

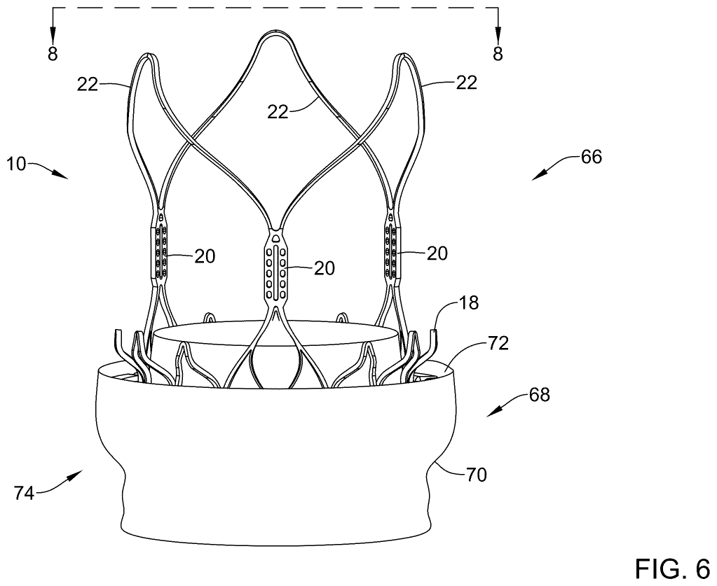

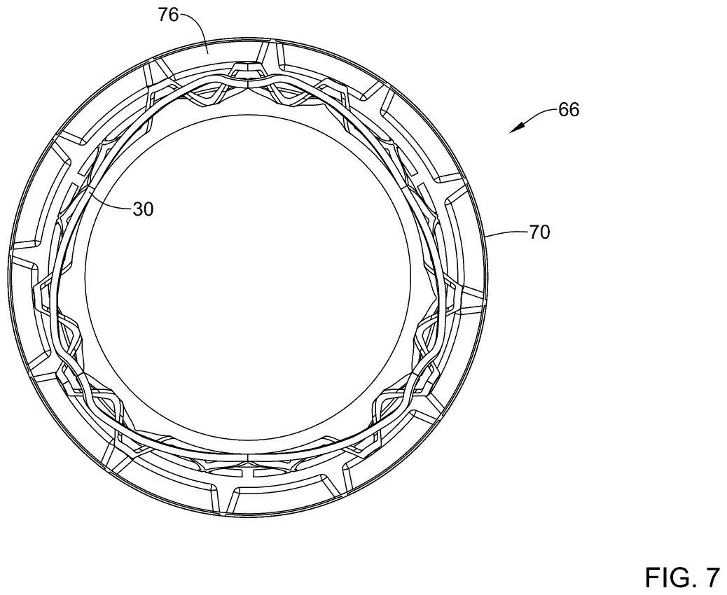

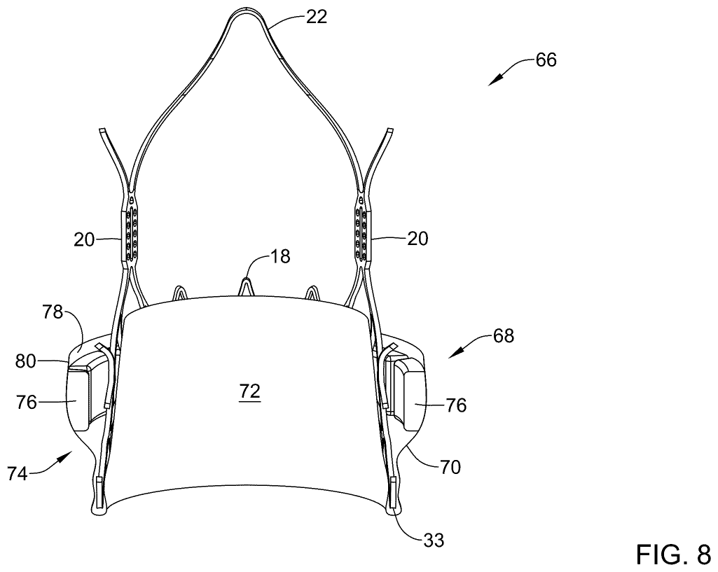

FIG. 6 is a side view of an illustrative replacement cardiac valve 66 that is assembled using the expandable frame 10 that was discussed with respect to FIG. 1. FIG. 7 is a top view of the replacement cardiac valve 66 and FIG. 8 is a cross-sectional view of the replacement cardiac valve 66 taken along the line 8-8 of FIG. 6. The replacement cardiac valve 66 includes a composite skirt 68 that includes an outer skirt 70 and an inner skirt 72. In some cases, the composite skirt 68 may be a polymeric fold-over skirt 74 that wraps around the lower extent 33 of the expandable frame 10 such that a portion of the polymeric fold-over skirt 74 extending outside of the expandable frame 10 forms the outer skirt 70 and a portion of the polymeric fold-over skirt 74 extending inside of the expandable frame 10 forms the inner skirt 72. FIG. 8 shows how the polymeric fold-over skirt 74 wraps or bends around the lower extent 33 of the expandable frame 10. In some cases, having the outer skirt 70 and the inner skirt 72 formed from a single sheet of polymer improves entrapment of fluids to reduce or eliminate the occurrence of paravalvular leak or replacement valve regurgitation. A variety of different polymers may be used to form the polymeric fold-over skirt 74. In some cases, the polymeric fold-over skirt 74 may be formed of PET (polyethylene terephthalate).

The replacement cardiac valve 66 includes a plurality of foam elements 76 that are disposed between the outer skirt 70 and the inner skirt 72. In some cases, the outer skirt 70 may be considered as including an inner surface 78 that faces towards the expandable frame 10 and an outer surface 80 that faces away from the expandable frame 10. In some cases, the foam elements 76 may each be secured to the inner surface 78 of the outer skirt 70. In some cases, the foam elements 76 may be adhesively secured to the inner surface 78 of the outer skirt 70. The foam elements 76 may each be printed onto the inner surface 78 of the outer skirt 70 and may extend towards the inner skirt 72. As can be seen in FIG. 7, the foam elements 76 extend circumferentially across individual struts of the plurality of struts 30 but do not extend into the diamond-shaped apertures or cells 32 (FIG. 1). As shown in FIG. 7, there is actually a clear space between the foam elements 76 and the individual struts of the plurality of struts 30. In some cases, the foam elements 76 are shown as being rectilinear, but other shapes and profiles are contemplated. The foam elements 76 may be distinct elements. In some cases, the foam elements may not be distinct elements, but may represent parts of a continuous foam band. In some cases, this can provide additional flexibility in compressing the expandable frame 10 when compressing the replacement cardiac valve 66 into its compressed configuration for delivery. When the replacement cardiac valve 66 is in its expanded configuration (as shown), the foam elements 76 help to push the outer skirt 70 into contact with the native annulus, and can help to seal around calcium deposits within the native annulus. Dimensions for the foam elements 76 may be selected to work with the particular dimensions of the expandable frame 10, for example. In some cases, the addition of the foam elements 76 may assist in better distributing forces from the expandable frame 10 that act on the anatomy, including the native annulus, and thus may reduce permanent pacemaker rates. In some cases, addition of the foam elements 76 may reduce migration of the replacement cardiac valve 66 by increasing the surface area by which pressure is applied to the anatomy by the replacement cardiac valve 66.

Hydrogel materials suitable for medical devices encompass several key categories. Natural polymer-based hydrogels include collagen, hyaluronic acid, fibrin, alginate, chitosan, and gelatin. In the synthetic polymer category, common materials are poly(ethylene glycol) (PEG), poly(vinyl alcohol) (PVA), poly(2-hydroxyethyl methacrylate) (PHEMA), poly(N-isopropylacrylamide) (PNIPAAm), poly(acrylic acid) (PAA), and poly(methacrylic acid) (PMAA). Composite or hybrid hydrogels combine different materials to create enhanced properties, such as PEG-collagen blends, PVA-chitosan composites, alginate-gelatin combinations, and PEG-hyaluronic acid networks. These materials possess essential properties that make them ideal for medical device applications, including biocompatibility, controlled degradation rates, tunable mechanical properties, high water content, ability to incorporate bioactive molecules, and controllable swelling behavior.

The foam used to create the foam elements 56 and 76 may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. In some instances, the expandable foam may be a shape memory foam. Suitable transition temperatures may be, for example, from 98.6° F. to 122° F. (37° C. to 50° C.), including from 98.6° F. to 104° F. (37° C. to 40° C.), which allows the shape memory foam to assume an initial configuration prior to and during delivery through a delivery catheter or other delivery device, and an expanded configuration for occlusion after delivery and release within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from 0.62 lb/ft³ to 62.4 lb/ft³ (10 kg/m³ to 1000 kg/m³), including from 0.62 lb/ft³ to 31.2 lb/ft³ (10 kg/m³ to 500 kg/m³), including from 0.62 lb/ft³ to 12.5 lb/ft³ (10 kg/m³ to 200 kg/m³), including from 1.25 lb/ft³ to 6.24 lb/ft³ (20 kg/m³ to 100 kg/m³).

Generally, the material for constructing the shape memory foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the shape memory foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the shape memory foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D, L-lactide, poly(beta-hydroxybutyrate), polygluconate including poly-D-gluconate, poly-L-gluconate, poly-D, L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerzation of various diketene acetals and diols; (d) polyanhydride homopolymers and copolymers such as poly(adipic anhydride), poly(suberic anhydride), poly (sebacic anhydride), poly(dodecanedioic anhydride), poly(maleic anhydride), poly[1,3-bis-(p-carboxyphenoxy)methane anhydride], and poly[alpha, omega-bis(p-carboxyphenoxy)alkane anhydride] such as poly[1,3-bis(p-carboxyphenoxy)propane anhydride] and poly[1,3-bis(p-carboxyphenoxy)hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the shape memory foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others.

A shape memory foam may have a thermal transition point (transition temperature) below which residual stress is maintained without a loading constraint. The thermal activation (which causes the shape memory) may be achieved with the desired material going through a semi-crystalline melt point or glass transition temperature between the first configuration and the expanded configuration. Several suitable thermal activation processes are known in the art and useful herein. In an example, the temperature activated memory shape foam may be formed for use as a medical device by first shaping a shape memory foam formed from a suitable material into its final expanded configuration; that is, the configuration that the shape memory shape foam will achieve once inserted into the left atrial appendage to provide the desired occlusive benefit. In this expanded configuration, the shape memory foam may generally have a diameter that will range from 0.39 inches to 1.97 inches (10 millimeters to 50 millimeters) and a length that will range from 0.39 inches to 1.97 inches (1 centimeter to 5 centimeters), although other diameters and lengths are within the scope of the present disclosure. Once this has been done, the foam may be heated above the transition temperature of the material; that is, the temperature at which a desired expansion will occur. As noted above, suitable transition temperatures may be from 98.6° F. to 122° F. (37° C. to 50° C.). Once the desired transition temperature has been achieved, the shape memory foam is held at a constant temperature and is re-shaped into an initial (unexpanded) configuration. This re-shaping is suitably done in a properly sized molding element and may be any suitable shape. In this collapsed configuration, the shape memory foam will generally have a diameter of less than 0.185 inches (4.7 millimeters) and a length that will range from 0.79 inches to 1.97 inches (2 centimeters to 5 centimeters), although other diameters and lengths are within the scope of the present disclosure. After insertion into the molding element, the temperature is reduced to a temperature below the transition temperature to set the new shape; for example, the temperature may be reduced to room temperature to set the new shape. Once this has been completed, the shape memory foam will remain in its first configuration until it is subjected to a temperature at or above the transition temperature, at which time it will expand into its expanded, or remembered, configuration. In some instances, exposure to water within the blood changes the glass transition temperature of the foam. As an example, the shape memory foam may have a dry Tg (glass transition temperature) that is above body temperature, and may have a wet Tg, after exposure to water, that is lower than body temperature.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “superelastic plateau” or “flag region” in its stress/strain curve like super-elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An implantable medical device adapted to be implanted at a treatment site, the implantable medical device comprising:
an expandable frame that is adapted to expand from a collapsed configuration to an expanded configuration, the expandable frame including:
an upper crown portion; and
a lower crown portion;
an inner skirt disposed along an inner portion of the lower crown portion of the expandable frame;
an outer skirt disposed along an outer portion of the lower crown portion of the expandable frame; and
an expandable member secured to the outer skirt.

2. The implantable medical device of claim 1, wherein the expandable member comprises a plurality of foam elements that are secured to the outer skirt such that the plurality of foam elements are disposed between the outer skirt and inner skirt.

3. The implantable medical device of claim 2, wherein the outer skirt includes an inner surface facing towards the lower crown portion of the expandable frame and an opposing outer surface, and the plurality of foam elements are printed onto the inner surface.

4. The implantable medical device of claim 1, wherein the inner skirt and the outer skirt are both part of a polymeric fold-over skirt.

5. The implantable medical device of claim 4, wherein the polymeric fold-over skirt is positioned folded over a lower extent of the lower crown portion of the expandable frame, with a portion of the polymeric fold-over skirt inside of the lower crown portion of the expandable frame forming the inner skirt and a portion of the polymeric fold-over skirt outside of the lower crown portion of the expandable frame forming the outer skirt.

6. The implantable medical device of claim 4, wherein the polymeric fold-over skirt comprises PET (polyethylene terephthalate).

7. The implantable medical device of claim 2, wherein the lower crown portion comprises a plurality of struts.

8. The implantable medical device of claim 7, wherein each of the plurality of foam elements extend radially beyond individual struts of the plurality of struts.

9. The implantable medical device of claim 7, wherein each of the plurality of foam spans circumferentially across individual struts of the plurality of struts.

10. A replacement cardiac valve adapted to be implanted within a native cardiac valve annulus, the replacement cardiac valve comprising:
an expandable frame that is adapted to expand from a collapsed configuration to an expanded configuration, the expandable frame including:
an upper crown portion; and
a lower crown portion including a plurality of struts;
a composite skirt wrapped over a lower extent of the lower crown portion, the composite skirt including an inner skirt disposed inside the lower crown portion and an outer skirt disposed outside of the lower crown portion; and
a plurality of foam elements secured to the outer skirt.

11. The replacement cardiac valve of claim 10, wherein the composite skirt comprises PET (polyethylene terephthalate).

12. The replacement cardiac valve of claim 10, wherein the plurality of foam elements are disposed between the inner skirt and the outer skirt.

13. The replacement cardiac valve of claim 10, wherein the plurality of foam elements are adapted to remain outside of the plurality of struts of the lower crown portion.

14. The replacement cardiac valve of claim 10, wherein the plurality of foam elements are adapted to extend into spaces between struts of the plurality of struts of the lower crown portion.

15. A replacement cardiac valve adapted to be implanted within a native cardiac valve annulus, the replacement cardiac valve comprising:
an expandable frame that is adapted to expand from a collapsed configuration to an expanded configuration;
an inner skirt surrounding a portion of the expandable frame;
an outer skirt surrounding a portion of the expandable frame; and
a plurality of foam elements secured to the outer skirt.

16. The replacement cardiac valve of claim 15, wherein the expandable frame comprises:
an upper crown portion;
a lower crown portion including a plurality of struts;
commissural posts extending proximally from upper crown portion; and
stabilization arches extending proximally from commissural posts.

17. The replacement cardiac valve of claim 15, wherein the outer skirt comprises an inner surface facing the expandable frame and an outer surface facing away from the expandable frame, and the plurality of foam elements are secured to the inner surface of the outer skirt.

18. The replacement cardiac valve of claim 15, wherein the outer skirt comprises a polymeric outer skirt.

19. The replacement cardiac valve of claim 15, wherein the outer skirt comprises PET (polyethylene terephthalate).

20. The replacement cardiac valve of claim 15, wherein the plurality of foam elements extend towards the inner skirt but are not attached to the inner skirt.
